(19) United States
(12) Patent Application Publication
(10) Pub. No.: US 2025/0205430 A1
(43) Pub. Date: Jun. 26, 2025

(54) CARTRIDGE ASSEMBLY AND FIXING MEMBER

(71) Applicant: Sanofi, Paris (FR)

(72) Inventors: Michael Helmer, Frankfurt am Main (DE); Reinhard Rieger, Obertshausen (DE); Martin Vitt, Frankfurt am Main (DE)

(21) Appl. No.: 18/848,026

(22) PCT Filed: Mar. 15, 2023
(86) PCT No.: PCT/EP2023/056535

§ 371 Date: Sep. 17, 2024

(30) Foreign Application Priority Data
Mar. 17, 2022 (EP) 22315069.9

(51) Int. Cl. A61M 5/31

(52) U.S. Cl. CPC A61M 5/3129; A61M 2207/00

(57) ABSTRACT

The present disclosure relates to a cartridge assembly provided with a fixing member for fixing a medicament container inside a housing component of a drug delivery device, wherein the housing component comprises a housing sidewall confining a container receiving space extending in a longitudinal direction (z) and wherein the fixing member comprises:
a fastening portion, and
a fixing structure.

### CROSS-REFERENCE TO RELATED APPLICATIONS

This is a United States National Stage Application, filed under 35 U.S.C. § 371, of International Patent Application No. PCT/EP2023/056535, which was filed on Mar. 15, 2023 and claims priority to European Patent Application No. 22315069.9, filed on Mar. 17, 2022, the disclosures of which are incorporated herein by reference in their entireties.

### TECHNICAL FIELD

The present disclosure relates to a cartridge assembly, particularly a cartridge assembly for a drug delivery device, such as an injection device and/or a pen-type injector. The present disclosure further relates to a fixing member for use with a cartridge or cartridge assembly, to a drug delivery device and to a method of assembling a cartridge assembly.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump. With respect to reusable injection or delivery devices, a patient may have to load or to replace a cartridge. Reusable injection devices typically comprise a multi-component housing. For instance, the housing may comprise a proximal housing component, such as a body and a distal housing component, such as a cartridge holder detachably connectable to the body. Once a medicament provided in a medicament container, such as a cartridge, is empty, the cartridge holder may be disconnected from the body of the injection device and the empty cartridge may be removed and replaced with a new cartridge.

Another concern that may arise from cartridges being manufactured in essentially standard sizes and manufactured to comply with certain recognized local and international standards. Consequently, such cartridges are typically supplied in standard sized cartridges (e.g. 3 ml cartridges). Therefore, there may be a variety of cartridges supplied by a number of different suppliers and containing a different medicament but fitting a single drug delivery device. As just one example, a first cartridge containing a first medicament from a first supplier may fit a drug delivery device provided by a second supplier. As such, a user might be able to load an incorrect cartridge or medicament into a drug delivery device and, then, dispense said medicament (such as a rapid or basal type of insulin) without being aware that the medical delivery device was perhaps neither designed nor intended to be used with such a cartridge.

As such, there is a growing desire from users, health care providers, caregivers, regulatory entities, and medical device suppliers to reduce the potential risk of a user loading an incorrect drug type into a drug delivery device. It is also desirable to reduce the risk of dispensing an incorrect medicament (or the wrong concentration of the medicament) from such a drug delivery device.

Some approaches to avoid unintended cross use of cartridges with non-intended drug delivery devices include a mechanical fixing of a cartridge to a housing component of the drug delivery device, such as a cartridge holder. The cartridge holder in turn may be then detachably or non-detachably connectable to a dose mechanism of such drug delivery devices.

Cartridges configured for insertion into a cartridge holder of a drug delivery device typically comprises a tubular-shaped main body merging into a radially narrowed neck, which in turn merges into a distally located and radially widened head portion. The head portion is typically covered by a septum retainer, e.g. made of a rather pliable material, such as an aluminum cap.

The septum retainer is configured to fasten a pierceable septum at a distal end of the head portion. With some approaches for fixing a cartridge in a cartridge holder the cartridge holder comprises a radially inwardly protruding structure, which is configured to engage the radially narrowed neck portion and/or to grip under the beaded cap of the cartridge. With such approaches and upon insertion of the cartridge into the cartridge holder the septum retainer may collide with a protruding structure of the cartridge holder, which may then lead to mechanical and/or visual defects in the region of the septum retainer of the cartridge.

It is therefore desirable to provide an improved cartridge assembly, which provides a reliable and durable fixing of the cartridge in a cartridge holder and which avoids damages or defects of the cartridge upon insertion of the cartridge into the cartridge holder.

### SUMMARY

In one aspect the disclosure relates to a fixing member for fixing a medicament container inside a housing component of a drug delivery device. With some examples the housing component is implemented as a cartridge holder. The medicament container may be implemented as a cartridge. The housing component comprises a housing sidewall confining a container receiving space. With some examples the housing sidewall confines a cartridge receiving space. The housing sidewall extends in a longitudinal direction (z), which may be also denoted as axial direction.

The fixing member comprises a fastening portion. The fastening portion comprises an inside surface complementary shaped to an outside surface of a counter fastening portion of the housing sidewall of the housing component. The inside surface is sized and shaped to support at least one of a longitudinally directed and a circumferentially directed fastening motion of the fastening portion relative to the counter fastening portion for reaching a final assembly configuration.

The fixing member further comprises a fixing structure. The fixing structure may be connected to the fastening portion. With some examples the fixing structure and the fastening portion are integrally formed, i.e. they are effectively immovably and/or permanently connected to each other.

The fixing structure comprises a first fixing element. The first fixing element is configured to extend into the container receiving space to mechanically engage with the medicament container located inside the container receiving space when the fastening portion of the fixing member reaches the final assembly configuration on the counter fastening portion.

With some examples the inside surface of the fastening portion and the outside surface of the counter fastening portion are complementary shaped in order to provide a sliding motion of the fastening portion relative to the counter fastening portion from a preassembly configuration, in which the fastening portion and the counter fastening portion may be engaged, into a final assembly configuration, in which the fixing structure of the fixing member mechanically engages with the medicament container located inside the container receiving space of the housing component.

This way and since the inside surface of the fastening portion matches with the outside surface of the counter fastening portion the fixing member is suitable for attaching and/or fixing to the outside of the medicament container. For providing a mechanical fixing of the medicament container inside the container receiving space it is no longer required to modify the inside structure or internal structure of the medicament container. By way of the fixing member attachable to the outside of the housing component and by way of the fixing structure extending into the container receiving space there can be provided a rather easy, straightforward, cost efficient and durable solution to fix and/or to retain a medicament container inside the container receiving space of the housing component.

By way of the fastening portion the fixing member can be fastened to an outside of the housing component of the drug delivery device. By way of the fixing structure the fixing member is enabled to mechanically interact with the medicament container when the medicament container is located inside the container receiving space of the housing component. The fixing structure may be configured to positively engage with an outside of the medicament container. It may engage with a recessed portion of the medicament container, e.g. with a neck portion of the medicament container.

According to a further example the fixing structure of the fixing member is configured to longitudinally engage with a recessed portion of the medicament container when the medicament container is located inside the container receiving space, e.g. in a final assembly position inside the container receiving space, and when the fixing member has reached a final assembly configuration or final assembly position on the housing component.

With some examples the fixing structure is configured to longitudinally abut with a complementary shaped counter fixing structure of the medicament container. This way, the medicament container can be retained and/or fixed in a final longitudinal position inside the container receiving space. When the fixing structure engages with the medicament container the medicament container is typically secured or fixed against a longitudinal movement relative to the housing component. When engaged with the fixing structure the medicament container may be secured against a proximally directed removal from the medicament container.

With some examples the fixing member is provided as a separate part configured for a releasable or non-releasable attachment to the housing component of the drug delivery device. This way, existing housing components or existing drug delivery devices can be retrofitted with a fixing member so as to provide a cartridge assembly comprising a housing component implemented as a cartridge holder, a medicament container implemented as a cartridge and comprising a fixing member as described herein, which is configured to fix the cartridge inside the cartridge receiving space of the cartridge holder.

By providing the fixing member as a separate part attachable to an outside of the housing component the medicament container can be assembled or arranged inside the container receiving space before the fixing member is attached or fastened to the housing component. This way, the inside of the container receiving space can be implemented to be void of any radially inwardly extending protrusions, that may otherwise harm the integrity, functionality or visual appearance of the medicament container.

Attaching the fixing member to the housing component or cartridge holder after insertion of the medicament container or cartridge inside the container receiving space provides the benefit to enable a rather easy and straightforward obstacle-less insertion of the medicament container inside the container receiving space. The fixing structure extending into the container receiving space and interacting with the medicament container for fixing the same at least with regard to the longitudinal direction can be assembled and attached to the housing component after insertion of the container in the container receiving space, thereby avoiding any negative impact on rather susceptible portions of the medicament container.

Since the inside surface of the fastening portion is complementary shaped to an outside surface of the counter fastening portion of the housing sidewall there may be provided a preassembly configuration, in which the fastening portion is mechanically attached to the counter fastening portion but in which preassembly configuration the fixing structure does not yet extend into the container receiving space.

The mutually corresponding sides of the outside surface of the counter fastening portion and the inside surface of the fastening portion may enable a premature fastening or prefixing of the fixing member to the housing component and may enable and support a sliding or gliding motion of the fixing member relative to the housing component towards and into a final assembly configuration, in which the fastening portion has reached a final assembly configuration on the counter fastening portion and in which the fixing structure mechanically engages with the medicament container.

In a preassembly configuration of the fixing member and the housing component the fixing structure may be located entirely outside the container receiving space. It may be only upon transferring the fixing member from the preassembly configuration or preassembly position towards and into the final assembly position or configuration that the fixing structure starts to extend into the container receiving space, e.g. such that the fixing structure assumes a longitudinal fixing position or fixing configuration with the cartridge when the fixing member approaches or reaches the final assembly configuration on the housing component.

In this way, the fixing member can be preassembled to the housing component, e.g. to a cartridge holder, before the medicament container is arranged inside the container receiving space. Hence, a separate handling of the fixing member and the medicament container can be avoided and is no longer required. By way of a preassembly of the fixing member to the medicament container the fixing member is readily provided on the medicament container and is already attached to the medicament container.

A user of the cartridge assembly or drug delivery device only has to manually insert a cartridge into the cartridge receiving space and to transfer the fixing member into its final assembly configuration or final assembly position, thereby establishing a mechanical interlock between the fixing member, the medicament container and the housing component.

According to further example the fastening portion comprises a sidewall of annular shape. Here, the complementary shaped counter fastening portion of the housing component may be also of annular shape. An annular shaped fastening portion may enable a longitudinal and/or a rotational sliding or gliding support for moving the fixing member and hence its fastening portion relative to the counter fastening portion for reaching the final assembly configuration. An annular shaped fastening portion is also beneficial to improve stability and rigidity of the fixing member.

The sidewall of the fixing member may be also of tubular shape. It may comprise a sleeve-like structure and may conform to a complementary shaped annular- or tubular-shaped outside surface of the counter fastening portion. The inner diameter of the sidewall of the fastening portion may closely match an outside diameter of a complementary shaped annular or tubular structure of the outside surface of the counter fastening portion. In this way there can be provided a friction fit between the fastening portion and the counter fastening portion.

With some examples the fastening portion may be clamped to the counter fastening portion, provided that the inner diameter of the sidewall of the fastening portion matches exactly an outer diameter or outer cross-section of the outside surface of the counter fastening portion. With some examples the sidewall of the fastening portion of the fixing member may be of conical shape. It may be complementary shaped to a respective conical shape of the counter fastening portion. By way of mutually corresponding conical shapes of the fastening portion and the counter fastening portion a kind of a press fit engagement between the fastening portion and the counter fastening portion can be provided.

According to a further example the fastening portion of the fixing member comprises an annular shaped fastening ring. The fastening ring may comprise a circumferentially closed structure. Such a structure is comparatively stable, robust and rigid. The annular shaped fastening ring allows and supports a longitudinal fastening motion as well as a rotational fastening motion for moving the fastening portion from a preassembly position or preassembly configuration towards and into the final assembly position or final assembly configuration.

With a further example the fastening portion comprises a closed ring. With other examples the fastening portion comprises a segmented or interrupted ring. A closed ring provides a high degree of dimensional stability and may provide a durable clamping or fastening of the fastening portion to the counter fastening portion. With a closed ring, there may be required a comparatively high assembly force, e.g. to urge the closed ring into a press fit configuration with the counter fastening portion.

With an open ring structure, the fastening portion comprises a comparatively high degree of flexibility. With an open ring structure, i.e. with a ring that is interrupted by a slit or through opening in its annular body the ring may be easily resiliently deformable in radial direction so as to vary its diameter. This way, the fastening portion could be easily adapted to varying geometries or to geometric tolerances of the counter fastening portion of the housing component.

An open ring structure of the fastening portion may provide a rather easy detachment or disassembly of the fixing member and the medicament container. With either implementation, i.e. with the fastening portion comprising a closed ring structure or an open ring structure the fixing member may be either undetachably or detachably fixable or attachable to the housing component.

According to a further example of the fixing member the inside surface of the fastening portion comprises a gliding surface, which is configured to glide on a complementary shaped counter gliding surface of the outer surface of the counter fastening portion along at least one of the longitudinal direction and the circumferential direction. With some examples the preassembly position of the fixing member may be located at a longitudinal offset from the final assembly configuration.

With other examples the preassembly configuration or preassembly position of the fixing member may be circumferentially offset or may be at a rotational offset from the final assembly configuration with a longitudinal axis of the container receiving space as an axis of rotation. With further examples the final preassembly position may distinguish from the final assembly position by a longitudinal and a rotational offset.

Providing of a gliding surface and a counter gliding surface on the mutually engaging inside and outside surfaces of the fixing member and the housing component there can be provided a well-defined fastening motion for transferring the fixing member from the preassembly position towards and into the final assembly position. With some examples the gliding surface and the complementary shaped counter gliding surfaces allow and support a rather easy and smooth sliding or gliding of the fixing member relative to the housing component.

With some examples the gliding surface and the complementary shaped counter gliding surface may provide, enable or define a helical gliding or sliding motion of the fixing member relative to the housing component in order to move the fixing member from the preassembly configuration towards and into the final assembly configuration.

With further examples the mutually corresponding gliding and counter gliding surfaces also enable a well-defined transfer of the fixing member from the final assembly configuration towards and into the preassembly configuration or towards and into a disassembly configuration, in which the fixing member is detached from the housing component.

According to a further example the first fixing element is at least one of resiliently movable or resiliently deformable relative to the fastening portion with regard to a radial direction. The fixing member and the fastening portion may be integrally formed. Here, the fixing member may comprise a single or unitary component, wherein the fastening portion is a first section and wherein the fixing structure or fixing element is a second section integrally formed with the first section.

By way of a resiliently movable or resiliently deformable fixing element there can be provided a kind of a snap fitting for snap-based fixing of the medicament container inside the container receiving space when the fixing member reaches the final assembly configuration. With some examples the housing component comprises a recess, e.g. a through recess in the housing sidewall, which overlaps or aligns with the fixing element when the fixing member reaches the final assembly configuration on the housing component, i.e. when the fastening portion reaches the final assembly configuration relative to the counter fastening portion.

As long as the fastening portion is not yet in the final assembly configuration relative to the counter fastening portion the fixing element of the fixing structure may be out of alignment or may be out of engagement with the recess or through opening as provided in the housing sidewall. In this preassembly configuration the fixing structure may be effectively hindered to extend into the container receiving space, e.g. in a radial inward direction.

It may be only upon reaching the final assembly configuration that the first fixing element of the fixing structure aligns or overlaps with a complementary shaped recess or through opening in the housing sidewall. The fixing member may then penetrate or enter the through opening in the housing sidewall so as to engage with the medicament container located inside the container receiving space. Here and when reaching the final assembly configuration the fixing member may resiliently relax into an initial or natural configuration, in which the fixing element extends into the container receiving space, thereby engaging with the medicament container located therein.

According to a further example the first fixing element is biased radially inwardly. The fixing element may be resiliently movable or may be resiliently deformable radially outwardly against an inherent restoring force. The fixing element may be implemented as a spring. It may comprise a leaf spring or the like spring arrangement, which has a tendency to deform or to move radially inwardly with regards to the annular shape of the fastening portion. In this way and with a radially inwardly biased first fixing element there can be provided a kind of a self-locking arrangement for the medicament container inside the container receiving space. Here, and as soon as the fixing element is moved into a final assembly configuration or final assembly position the fixing element may be allowed to deform or to move radially inwardly so as to enter or to extend into the container receiving space from outside the housing sidewall thereby mechanically engaging with the medicament container located inside the container receiving space.

According to a further example the first fixing element protrudes radially inwardly from the inside surface of the fastening portion. The first fixing element may be elastically deformable or resiliently deformable radially outwardly, e.g. when the fixing member is in the preassembly configuration on the outside surface of the counter fastening portion. When reaching the final assembly configuration, e.g. in which the fixing structure overlaps or aligns with a through opening in the housing sidewall, the fixing element is allowed to protrude radially inwardly through the respective through opening or recess in the housing sidewall. It may then protrude radially inwardly to such an extent that the fixing element protrudes radially inwardly not only from the inside surface of the fastening portion but also radially inwardly from an inside surface of the housing sidewall confining the container receiving space.

In this way, the fixing element or at least a portion thereof may protrude radially inwardly from outside the housing sidewall into the container receiving space, thereby mechanically interacting with the medicament container located inside the container receiving space.

According to a further example the first fixing element comprises a snap element with a tongue portion extending in the longitudinal direction. The tongue portion may extend in longitudinal distal direction from the fastening portion. The tongue portion, in particular a distally located distal free end of the tongue portion may protrude radially inwardly from the annular shape of the fastening portion.

This way, the inside of the fastening portion may engage with an outside surface of the housing sidewall while the fixing element protrudes radially inwardly into the container receiving space. The first fixing element may comprise a rather straight, or elongated shape extending at a certain angle relative to the longitudinal direction. It may extend radially inwardly towards the distal end of the housing component when correctly assembled to the housing component.

With other examples the first fixing element may comprise at least in sections a beveled shape or curved shape, e.g. in order to provide a well-defined holding or fixing force for the medicament container inside the container receiving space.

In a further example the first fixing element comprises a snap element with a tongue portion extending in the longitudinal direction. The tongue portion may extend in longitudinal and radial direction as seen from the fastening portion. With some examples the snap element or tongue portion may extend in distal direction from the fastening portion so as to engage with a proximally facing counter stop face of the medicament container.

In this way, the snap element or tongue portion, e.g. engaging with a radially recessed portion of the medicament container may be configured to prevent a proximally directed movement of the medicament container relative to the housing component.

With some examples the fastening portion may comprise a proximal end face, e.g. a stop face that faces in proximal direction so as to abut or to engage with a complementary stop face of the housing sidewall facing in distal direction. This allows and supports to mount the fixing member onto the housing component along a longitudinal proximal direction until the proximal end face of the fixing member engages the complementary shaped distal stop face of the housing component or housing sidewall. The distally extending snap element or tongue portion of the fixing element may then engage in longitudinal direction with a proximally facing counter stop face of the medicament container, e.g. provided at or near a transition from a radially widened head into a radially stepped down neck portion of the medicament container.

In this way the medicament container can be fixed against a proximal movement through a longitudinal abutment between the proximally facing counter stop face with a distally facing or distally protruding snap element or tongue portion of the fixing element. When longitudinally engaged with the medicament container, the fixing element, e.g. unitarily shaped or integrally formed with the fastening portion or fixed to the fastening portion at least with regards to the longitudinal director may transfer a respective proximally directed force effect onto the fastening portion, which in turn is in longitudinal proximally facing abutment with a distally facing stop face of the medicament container.

In this way the medicament container can be secured against a proximal displacement relative to the housing component. By way of the fixing member the medicament container can be mechanically locked inside the container receiving space of the housing component.

With typical examples, the housing component comprises an insert opening at its proximal end, that provides a distally directed longitudinal insertion of the medicament container into the container receiving space. The container receiving space of the housing component may be confined in distal direction by an end wall providing a distal stop for the distal end of the medicament container.

With further examples there may be provided a twofold and mutual fixing of the fixing member and the container inside the housing component. As described above, the fixing member may serve to fix the medicament container inside the container receiving face. With further examples it is also conceivable that the mutual mechanical engagement of the fixing structure of the fixing member with e.g. a recessed portion of the medicament container inside the container receiving space serves to fix the fixing member on the outside surface of the counter fastening portion.

Since the medicament container may be in longitudinal abutment with a distal end wall of the medicament container it may be hindered to move in distal direction relative to the medicament container. Since the snap element or tongue portion of the fixing element is in longitudinal abutment with the recessed portion, e.g. with the neck portion or with an radial undercut of a portion of the medicament container and hence when the fixing member is in longitudinal abutment with a proximally facing counter stop face as provided on the medicament container the fixing member is hindered to move in distal direction relative to the housing component.

This way, the mutual longitudinal abutment or longitudinal engagement of the first fixing member of the fixing structure with the medicament container serves to lock the fixing member onto the housing component. In this way, the fixing member may be permanently and non-detachably fixed to the housing component and to the medicament container located therein. In this way the fixing member and the medicament container mutually interlock to the housing component such that neither the fixing member nor the medicament container can be removed therefrom once the fixing member has reached the final assembly position.

According to a further example the tongue portion of the first fixing element comprises a bottom portion and a head portion. The bottom portion and the head portion are separated along the longitudinal direction. The bottom portion is connected to the fastening portion and the head portion forms a free end section of the tongue portion. Typically, it is predominately the head portion that is radially movable relative to the fastening portion of the fixing member. It may be exclusively the head portion that protrudes radially inwardly from the sidewall of the fastening portion whereas the bottom portion of the tongue portion is permanently fixed or integrally formed with the fastening portion.

The tongue portion may be of rather straight elongated shape and may extend at a predefined angle radially inwardly from the annular sidewall of the fastening portion. As seen in circumferential direction the tongue portion may be tapered towards its free end. Hence, a tangential or circumferential width of the tongue portion towards or near the head portion may be smaller than towards or near the bottom portion.

With other examples the tongue portion may at least in sections comprise one of a beveled section, a bulged section or arched section. With some examples it may comprise a S-like or C-like shape in order to provide a rather smooth radial mechanical engagement with the recessed portion of the medicament container, e.g. with the neck portion of the medicament container.

With some examples the fixing member may be assembled to the housing component in the final assembly configuration before the medicament container is inserted into the container receiving space. Here, the tongue portion of the first fixing element may radially inwardly protrude into the container receiving face. As the medicament container is inserted into the container receiving space, e.g. in longitudinal direction towards the distal end of the container receiving space, a distal end of the medicament container may engage with the radially inwardly extending tongue portion and may urge the tongue portion radially outwardly against the resilient restoring force as provided by the first fixing element.

When the medicament container reaches a final assembly position or final assembly configuration inside the container receiving space the tongue portion may relax into its initial unbiased configuration, thereby engaging with the neck portion of the medicament container so as to fix or to immobilize the medicament container to the housing component at least with regards to the longitudinal direction.

According to a further example the first fixing element comprises a distally facing stop face to engage with a proximally facing counter stop face of the medicament container. With some examples the proximally facing counter stop face of the medicament container is provided at or near a distal end of the medicament container. It may be provided in a transition zone between a radially widened head portion merging into a radially narrowed neck portion towards the proximal direction.

The radially inwardly protruding first fixing element may longitudinally engage or axially engage with a stepped down section of the head portion facing towards the proximal direction when the medicament container reaches a final assembly configuration. In this way, the first fixing element provides a proximal end stop for the medicament container and may prevent a proximally directed movement of the medicament container relative to the housing component when the medicament container has reached the final assembly position.

According to a further example the first fixing element comprises an inside facing surface to engage with an outside surface of a flap portion of a counter fixing structure of the housing. The flap portion is at least one of movable or deformable in radial direction. The first fixing element is configured to urge the flap portion radially inwardly when reaching the final assembly configuration. Here, the flap portion may be located radially between the first fixing element and the medicament container. The flap portion may be integrally formed with the housing component. It may be made from the same material than the housing component. Typically, the flap portion may comprise a plastic material, e.g. an injection molded plastic material. Also, the housing component may comprise a plastic material, e.g. an injection molded plastic material.

The flap portion may be movably connected to the housing component, e.g. by a film hinge. The flap portion may provide a mechanical damper or cushion for the medicament container so as to prevent a direct mechanical contact between the first fixing element of the fixing member and an outside surface of the medicament container. With some examples the medicament container comprises a vitreous barrel, e.g. a barrel made of glass. Such a barrel may be rather susceptible to mechanical shock. With some examples the fixing member itself is made of an injection molded plastic component. With other examples the fixing member comprises a metal or is made of metal or a metal alloy. Use of a metallic fixing member is beneficial to provide a high degree of mechanical stability as well as to provide a comparatively high mechanical restoring or fixing force, e.g. for the first fixing element, while keeping the geometric dimensions of the fixing member rather small.

The flap portion of the counter fixing structure of the housing sidewall may act as or may provide a mechanical shock absorber. Its outside facing surface is configured to engage with an inside facing surface of the first fixing element. It may be only or exclusively an inside facing surface and/or a longitudinal end face of the flap portion that may get in direct mechanical engagement with the medicament container.

According to a further example the first fixing element comprises a side edge extending in longitudinal direction and protruding at least in sections from the inside surface of the fastening portion to engage with a recess of the counter fixing structure on the outside surface of the housing sidewall. Further and by way of the side edge of the fixing element there can be provided a rotational interlock between the fixing member and the housing component. The recessed counter fixing structure on the outside surface of the housing sidewall may comprise a through opening, through which the first fixing element may extend in radial direction. In such a configuration the side edges of the first fixing element may be located between complementary shaped side edges of the counter fixing structure as seen in circumferential or tangential direction.

Typically, the inner circumference of the counter fixing structure, e.g. a circumferential or tangential distance between the edges of the counter fixing structure of the housing sidewall is larger than or equal to an outside circumferential distance between opposite side edges of the longitudinally extending fixing element. Accordingly, the fixing element will be able to engage the counter fixing structure, thereby extending into and/or through the recess counter fixing structure, thereby providing or establishing a rotational interlock between the fixing member and the housing component.

According to a further example the fixing structure further comprises a second fixing element and/or a third fixing element located circumferentially offset from the first fixing element and configured to extend into the container receiving space to mechanically engage with the medicament container inside the container receiving space. Typically, the first fixing element and the second fixing element are somewhat or expediently identical. The same may apply to the optional third fixing element.

The first, the second and/or the third fixing element may be equidistantly spaced along the circumferential direction of the fastening portion. With a first and a second fixing element, the first and the second fixing elements may be located at diametrically opposite to each other. When there are provided there fixing elements, they may be placed at an angular distance of 120° from each other. When the fixing member comprises two or more fixing element also the housing component comprises a respective number of matching recesses of the counter fixing structure, e.g. a respective number of recesses or through openings in the housing sidewall so as to engage with the respective fixing element.

By providing at least a second and/or a third fixing element, there can be provided a twofold or even threefold mechanical fixing between the fixing member, the housing component and the medicament container located therein. With an increasing number of fixing members, there can be provided a redundant fixing or interlocking configuration for keeping and/or fixing the medicament container inside the container receiving space.

According to a further aspect the disclosure relates to a cartridge assembly for a drug delivery device, in particular to a cartridge assembly for an injection device, such as a handheld pen-type injector. The cartridge assembly comprises a cartridge. The cartridge comprises a tubular-shaped barrel containing a medicament. The cartridge, in particular its tubular barrel comprises a radially recessed neck portion. Typically, the cartridge comprises a stopper by way of which the cartridge can be sealed towards a proximal end. The stopper is movably disposed inside the barrel for expelling the medicament through a dispensing end of the barrel. Typically, the dispensing end is provided at the distal end of the barrel. The proximal end of the barrel is sealed by the movable stopper.

The cartridge assembly further comprises a housing component of the drug delivery device. The housing component may be implemented as a cartridge holder. Typically, the housing component is configured and shaped to receive or to accommodate the cartridge. The housing component comprises a housing sidewall. The housing sidewall confines a cartridge receiving space extending in a longitudinal direction. It is sized to receive the cartridge. Typically, the cartridge receiving space is sized to receive the cartridge in its entirety.

The housing component further comprises a counter fastening portion, which is provided on an outside surface of the housing sidewall. The cartridge assembly further comprises a fixing member as described above. The fastening portion of the fixing member is mechanically engageable with the counter fastening portion of the housing component. When the fixing member is engaged with the counter fastening portion the first fixing element of the fixing structure of the fixing member extends into the container receiving face and mechanically engages with the neck portion of the cartridge.

Typically, by way of the fastening portion the fixing member is attachable or connectable to the outside surface of the housing sidewall. By way of the first fixing element there is provided a mechanical interaction between the fixing member and the cartridge located inside the container receiving space. With some examples the fixing member protrudes radially inwardly into the container receiving space, thereby positively engaging with the radially recessed neck portion of the cartridge. In this way, the cartridge can be secured and/or fixed inside the container receiving space at least with regards to the longitudinal direction.

Since the cartridge assembly is equipped with a fixing member as described above, all features, effects and benefits as described above in connection with the fixing member equally apply to the cartridge assembly; and vice versa.

It should be noted that a cartridge as described herein is a particular example of a medicament container filled with a medicament, e.g. a liquid medicament. Insofar, each reference to a cartridge or cartridge receiving space is a synonym for a medicament container or container receiving space as mentioned herein.

According to a further example the housing sidewall of the housing component comprises a counter fixing structure. The counter fixing structure comprises a first through opening extending through the housing sidewall. The first through opening is sized to receive the first fixing element radially therein or therethrough. In this way, the fixing element or at least a portion thereof is enabled to reach through the housing sidewall. It may extend from outside the housing sidewall through the housing sidewall and into the interior of the container receiving space. Typically and when implemented as a snap element with a tongue portion a free end of the tongue portion extends through the through opening and will be arranged inside the container receiving space so as to mechanically engage with the radially recessed portion of the medicament container, e.g. with a portion of a cartridge. With the first fixing element extending radially through the first through opening there may be also provided a longitudinal and/or a rotational interlock between the fixing member and the housing component. A longitudinally extending side edge or oppositely located longitudinal side edges of the first fixing element may be located between respective side edges of the through opening, thereby preventing a rotational motion of the fixing member relative to the housing component once the first fixing element engages with the first through opening.

With further examples there may be provided numerous through openings along the circumference of the housing sidewall. The number and size or individual through openings may be provided in correspondence to the number and geometry or size of the fixing elements of the fixing member. With some examples, wherein the fixing member comprises a first and a second fixing element, also the housing component comprises a counter fixing structure with a first through opening and with a second through opening. The size and the position of the first and second through openings relative to each other matches with the size and position or orientation of the first and second fastening element relative to each other.

With some examples the counter fixing structure is provided at or near a neck portion of the housing component. It may be provided at or near a distal end of the housing component, which may be further provided with a needle connector. The needle connector may comprise a threaded interface for detachably fastening a needle assembly to the housing component.

According to a further example the housing sidewall comprises a counter fixing structure with a first flap portion movable or deformable in radial direction relative to the housing sidewall. The first flap portion comprises an outside surface to engage with the first fixing element of the fixing structure of the fixing member. The first flap portion further comprises an inside surface to engage with the neck portion of the cartridge. Typically, the first flap portion serves as a mechanical shock absorber and/or as a sandwich element located between the outside surface of the medicament container and an inside surface of the first fixing element of the fixing structure of the fixing member. In this way, a direct mechanical contact between the first fixing element and an outside surface of the medicament container can be avoided. This allows to implement the fixing member as a metal component, which may have otherwise have a certain potential to harm the integrity of the medicament container.

The first flap portion may be integrally attached to and/or may be integrally formed with the housing sidewall. The first flap portion may be rotatable relative to the sidewall along a pivot axis extending perpendicular to the longitudinal direction. The pivot axis may be integrated into or may be located inside the housing sidewall. The flap portion may be of rectangular shape. Only one side edge of the rectangularly shaped flap portion may be connected with the housing sidewall. Further side edges of the flap portion may be separated from the sidewall by a U-shaped slit.

According to a further example the counter fastening portion of the housing component comprises a tubular-shaped sidewall portion with an outer gliding surface enabling or supporting at least one of a longitudinal and a circumferential sliding or gliding motion of the fastening portion of the fixing member relative to the housing sidewall. An outer gliding surface enables a rather smooth and easy sliding or gliding movement of the fixing member, e.g. for transferring the fixing member from a preassembly configuration, in which the fastening portion thereof is not yet engaged with the counter fastening portion of the housing sidewall, into a final assembly configuration, in which the fastening portion of the fixing member is engaged with the counter fastening portion of the housing sidewall.

Moreover, in the preassembly configuration the fixing structure and hence the first fixing element is not yet engaged with the counter fixing structure and/or with the medicament container located inside the container receiving space. It is only upon transferring the fixing member into the final assembly configuration or final assembly position, that the fixing structure and hence the first fixing element thereof engages with the counter fixing structure and/or with the medicament container for fixing the same inside the container receiving space at least with regards to the longitudinal direction.

According to a further example the fixing member comprises a proximal or proximally facing end face configured to longitudinally abut with a distally facing stop face of the housing sidewall. In this way, an assembly motion of the fixing member directed towards the proximal direction can be delimited. Moreover, a final assembly configuration or final assembly position of the fixing member, in particular of its fastening portion to the counter fastening portion can be defined by a mutual longitudinal abutment of the proximally facing end face of the fixing member with the distally facing stop face of the housing sidewall.

The distally facing stop face of the housing sidewall may be provided by a radial step, e.g. by a flange portion. In this way, and when the proximal end face of the fixing member engages the distally facing stop face of the housing sidewall the fixing member is blocked against any further proximally directed sliding motion relative to the housing sidewall. This is of particular benefit to provide a proximal stop for the fixing member. The fixing structure of the fixing member may in turn provide a distally facing stop to engage with the radially recessed portion of the medicament container. Here, a proximally facing counter stop face of the medicament container, e.g. provided at the head portion of the medicament container may get in a longitudinal abutment with a distally facing stop face of the third fixing element and hence of the fixing structure.

In this way, a proximally directed force effect eventually acting on the medicament container, e.g. in an attempt to remove the medicament container from the housing component, is transferred into a respective proximally directed force effect present on the fixing member. The fixing member is in turn secured against any further proximally directed displacement relative to the housing sidewall by its proximally facing edge or stop face engaging the distally facing stop face of the housing sidewall. In this way, a proximally directed removal of the medicament container from the cartridge receiving space can be effectively blocked and prevented when the fixing member has reached its final assembly configuration and when the fixing element of the fixing structure engages the radially recessed portion of the medicament container.

According to a further example the radially recessed portion of the cartridge or medicament container is delimited in distal direction by a radially widened head portion. The head portion comprises a proximally facing counter stop face to longitudinally abut with at least one of the first fixing element and the counter fixing structure. With some examples and when the counter fixing structure of the housing sidewall is provided with a first flap portion the flap portion may comprise a distally facing stop face to engage with the proximally facing counter stop face of the head portion of the cartridge. Here, the fixing member, in particular its first fixing element serves to urge the flap portion radially inwardly, i.e. into the radially recessed portion of the cartridge, thereby engaging the distally facing stop face of the flap portion with the proximally facing counter stop face of the head portion of the cartridge.

With further examples, wherein the counter fixing structure of the housing sidewall is void of a movable flap portion and wherein the counter fixing structure comprises a through opening to receive the first fixing element it may be a distally facing free end of the radially inwardly protruding first fixing element that longitudinally abuts or engages the proximally facing counter stop face of the head portion of the cartridge to prevent a proximally directed movement or removal of the cartridge relative to the container receiving space or housing portion.

According to a further aspect the present disclosure also relates to a method of assembling a cartridge assembly of the drug delivery device. The cartridge comprises the steps of inserting a cartridge into the cartridge receiving space of a housing component of a cartridge assembly. Thereafter, a fixing member is arranged in a final assembly position on the counter fastening portion of the housing sidewall of the housing component and a first fixing element of the fixing member is mechanically engaged with a neck portion of the cartridge.

Typically, the method is to be executed or conducted by making use of a cartridge assembly as described above and by making use of a fixing member as described above. Insofar, all features, effects and benefits as described above equally apply to the method of assembling of the cartridge assembly; and the vice versa.

Typically, the mechanical engagement of the first fixing element of the fixing structure with the neck portion of the cartridge is automatically obtained by moving or transferring the fixing member from a preassembly position or preassembly configuration into a final assembly configuration of final assembly position. This final stage of assembly may be provided by a sliding motion of the fixing member, e.g. in longitudinal direction and/or in circumferential direction relative to the housing sidewall of the housing component. For this, the fastening portion of the fixing member and a complementary-shaped counter fastening portion as provided on an outside surface of the housing sidewall may be provided with complementary shaped gliding and counter sliding surfaces.

The presently proposed method of assembling a cartridge assembly provides a fixing of the cartridge or medicament container inside the container or cartridge receiving space of the housing component, which is typically implemented as a cartridge holder. Here, it is of particular benefit, that the fixing member in a preassembly position does not protrude inwardly into the container receiving space. As long as the fixing member is in the preassembly configuration the first fixing element may be located on the outside surface of the housing sidewall. It may be pretensed or pre-biased radially inwardly against the outside surface of the housing sidewall. It is only after insertion of the medicament container into the container receiving space that the fixing member is transferred into the final assembly configuration, thereby aligning the first fixing element with a through opening or counter fixing structure as provided on or in the housing sidewall of the housing component. Then, the resiliently movable or resiliently deformable fixing element may is allowed to move radially inwardly through the through opening of the housing sidewall and into a radially recessed portion of the cartridge for fixing the same with regards to the longitudinal direction.

In this way, a radially inwardly protruding portion of the cartridge assembly will be provided after insertion of the cartridge into the container or cartridge receiving space. During or for insertion of the cartridge into the cartridge receiving space the inside of the housing sidewall confining the container or cartridge receiving space may be void of any radially inwardly extending protrusions that could otherwise harm the integrity of the distal dispensing end of the medicament container.

With further examples it is conceivable to arrange the fixing member in the final assembly configuration before the cartridge is inserted into the cartridge or container receiving space. During the insert motion, the distal end of the cartridge or medicament container may at least temporally urge the radially inwardly protruding first fixing element in a radial outward direction in the course of a final assembly process. When reaching a final assembly position, i.e. when reaching the distal end position inside the container receiving space, the radially stepped down or radially recessed portion of the cartridge will align with the first fixing element, thereby allowing the first fixing element to snap or to latch radially inwardly to engage in longitudinal direction with the proximally facing counter stop face as provided on the radially widened head portion of the cartridge or medicament container.

According to another aspect the disclosure also relates to a drug delivery device, e.g. implemented as an injection device, such as a pen type in injector. The drug delivery device comprises a body, e.g. in form of a proximal housing component. The body or the proximal housing component is typically connectable to a cartridge assembly as described above. The drug delivery device typically comprises a drive mechanism, e.g. comprising a piston rod to exert a dispensing force onto a piston located inside the tubular shaped barrel of the cartridge, wherein the cartridge is typically provided and/or fixed inside the housing component or cartridge holder of the cartridge assembly. Typically, the cartridge assembly is configured to releasably or non-releasably fix to the body of the drug delivery device.

The drug delivery device typically comprises a cartridge assembly as described above. Insofar, all features, effects and benefits as described above with regards to the cartridge assembly equally apply to the drug delivery device.

With some examples of the drug delivery device or of the cartridge assembly the fixing member may be mechanically coded to the cartridge or to the type of a cartridge. Hence, there may be selected one of a plurality of available and differently configured fixing members in accordance to the size of the cartridge and/or in accordance to the type of medicament contained in the cartridge. Likewise, the counter fastening portion and/or the counter fixing structure of the housing sidewall may be encoded for a particular type of a cartridge or medicament.

With other examples, the fixing member may be universally usable with a large variety of different types of cartridges, different medicaments located in such cartridges and different types of housing components, e.g. cartridge holders. Here, a pharmaceutical manufacturer may ensure that a correct cartridge is always inserted into a suitable and matching cartridge holder. By making use of the fixing member as described herein, it can be ensured, that the cartridge is permanently and non-detachably fixed inside the cartridge receiving space of the cartridge holder or housing component. Then, a cartridge assembly comprising a cartridge, a housing component and a fixing member by way of which the cartridge is non-removably attached inside the cartridge holder or housing component may be commercially distributed. End users or patients will be effectively hindered to detach or to remove the cartridge from the cartridge holder or housing component.

With other examples, differently configured fixing members may distinguish from each other by at least one of a their fastening portion and their fixing structure. For instance, a first fixing member may be configured for engagement with only a first type of a cartridge. The first housing component may be mechanically coded to connect exclusively to only one of a number of available bodies or drive mechanisms of drug delivery devices. Accordingly, different encoded housing components or differently encoded cartridge holders may distinguish from each other by a shape and/or geometry of a cartridge receiving space, so as to avoid unintended cross use of a cartridge not intended for use with a particular drive mechanism or body of an injection device.

Cartridge assemblies comprising a cartridge, a cartridge holder and a fixing member, by way of which the cartridge is permanently fixed or is fixable inside the cartridge holder, can be manufactured and assembled by a pharmaceutical manufacturer. Such cartridges assemblies might be commercially distributed for reusable drug delivery devices. The cartridge holder of the cartridge assembly and the body of the drug delivery device may be mechanically encoded to prevent unintended cross use, i.e. to prevent use of a body and/or drive mechanism of a first type with a cartridge or cartridge holder of a second type, not intended for use with this particular body or drive mechanism. In effect and by providing cartridge assemblies with a cartridge fixed inside the drug delivery device patient safety can be enhanced and misuse of drug delivery devices can be effectively prevented.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber.

In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys(B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N-(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014(E). As described in ISO 11608-1: 2014(E), needle-based injection systems may be broadly distinguished into multi-dose container systems and single-dose (with partial or full evacuation) container systems. The container may be a replaceable container or an integrated non-replaceable container.

As further described in ISO 11608-1: 2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically shows an example of a drug delivery device,

FIG. 2 shows a cartridge holder and a cartridge for insertion into the cartridge holder,

FIG. 3 shows a cartridge assembly before attaching a fixing member to the cartridge holder,

FIG. 4 is a perspective view of the fixing member,

FIG. 5 is an enlarged view of the fixing member attached to the cartridge holder in the final assembly configuration,

FIG. 6 is a side view of the cartridge assembly with the fixing member attached to the cartridge holder,

FIG. 7 shows a further example of the fixing member in a preassembly configuration attached to the cartridge holder before insertion of the cartridge, shows the configuration of FIG. 7 after insertion of the cartridge into the cartridge FIG. 8 receiving space,

FIG. 9 shows the cartridge assembly of FIGS. 7 and 8 in the final assembly configuration,

FIG. 10 shows a longitudinal cross-section of the cartridge assembly in the final assembly configuration,

FIG. 11 is an enlarged view of the distal end of the cartridge assembly of FIG. 10,

FIG. 12 is a further perspective view of the distal end of the cartridge assembly according to FIG. 11,

FIG. 13 shows a further example of a cartridge assembly before inserting the cartridge into the cartridge holder,

FIG. 14 shows the fixing member attached to the cartridge holder in a preassembly configuration,

FIG. 15 is a cross-section through the distal end of the cartridge assembly, and

FIG. 16 is a flowchart of a method of assembling the cartridge assembly.

### DETAILED DESCRIPTION

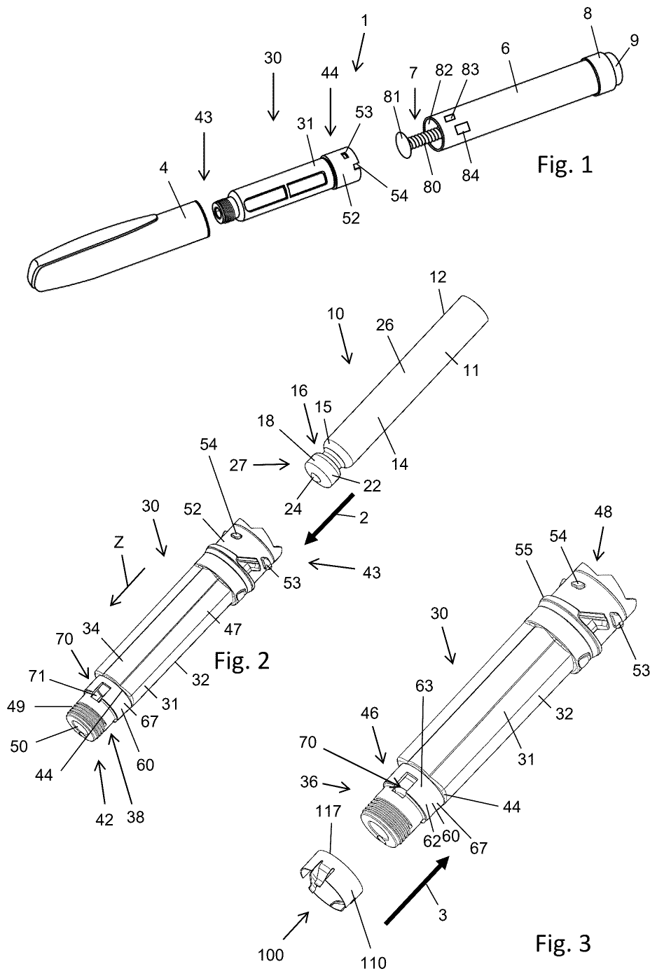

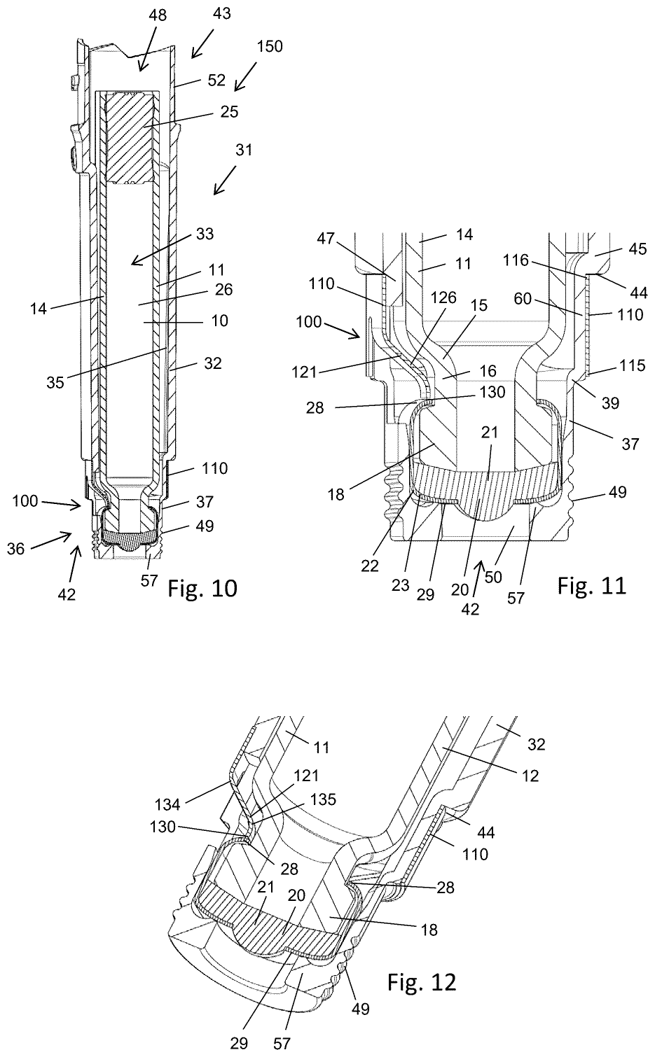

FIG. 1 shows an example of a drug delivery device 1 implemented as a pen-type injector. The drug delivery device 1 comprises numerous housing components. In detail the drug delivery device 1 comprises a tubular shaped housing component 30 implemented as a cartridge holder 31 sized to accommodate a medicament container 5, such as a cartridge 10, which is illustrated in FIG. 2. The drug delivery device 1 further comprises a body 6 forming or constituting a proximal housing component 6. The body 6 houses a drive mechanism 7. The drive mechanism 7 may comprise a dose member 8 and/or a trigger 9 for setting and/or dispensing a dose of a medicament 26 contained in a medicament container 5, which in the present case may be implemented as a cartridge 10. Typically, the drive mechanism 7 comprises a piston rod 80, e.g. provided with a pressure piece 81 at its distal end. The pressure piece 81 is configured to abut in longitudinal direction (z) with a proximal end face of a piston 25 slidably arranged inside the barrel 11 of the cartridge 10. The piston 25 is for instance illustrated in FIG. 10.

The barrel 11, which may be of a vitreous material, such as glass, comprises a sidewall 12 with a main portion 14 of substantially tubular shape. The piston 25 is slidably displaceable along the main portion 14. Towards the dispensing and 27 the main portion 14 merges into a narrowing shoulder portion 15, which in turn merges in to a radially stepped down neck portion 16. The neck portion 16 terminates with a head portion 18, which, at least on its outside surface may comprise a somewhat radially widened cross-section compared to the neck portion 16.

The drug delivery device 1 further comprises a protective cap 4 configured to cover the dispensing end 27 of the drug delivery device 1. Typically, the housing component 30 is implemented as a cartridge holder 31. The housing component 30 comprises a distal end 43 and a proximal end 44. The distal end 43 is configured to engage with a needle assembly, e.g. a double-tipped injection needle by way of which a dose of the liquid medicament 26 can be injected into biological tissue. The distal end 43 may comprise a cylindrical portion 38. The cylindrical portion 38 may comprise or constitute a needle connector 36 with a threaded section 49. The threaded section 49 is one example to support a threaded connection with a complementary shaped needle hub equipped with the injection needle.

The distal end 43 of the cartridge holder 31 further comprises an aperture 50 to receive a proximal portion or a proximally extending portion of the double-tipped injection needle. The injection needle is configured to pierce and/or to penetrate a seal 20 at the distal end of the cartridge 10. The seal 20 may comprise a pierceable septum 21, which is fixed to a distally located head portion 18 of the cartridge by way of a septum retainer 22. The septum retainer 22 may comprise a beaded cap 23, e.g. made of a pliable material, such as aluminum or plastic.

The septum retainer 22 comprises an aperture 24 that aligns with the aperture 50 when the cartridge 10 is received in the interior of the cartridge holder 31. Inside the cartridge holder 31 there is provided a cartridge receiving space 33, which is effectively occupied by the cartridge 10 when assembled inside the cartridge holder 31. The cartridge holder 31, in particular the housing sidewall 32 comprises a main portion 47 comprising a somewhat cylindrical shape. The main portion 47 is adapted to receive the main portion 14 of the barrel 11 of the cartridge 10.

The cartridge holder 31 comprises a proximal end 44, which may be provided with an insert 52 sized and configured for insertion into a receptacle 82 as provided at the distal end of the body 6. Towards the distal direction 2, the insert 52 is delimited by a flange portion 55. When the cartridge holder 31 is attached to the body 6, the flange portion 55 may be in longitudinal abutment with a distal end face of the sidewall of the receptacle 82.

On the inside of the receptacle 82 there may be provided a counter fastening structure 83, e.g. in form of a radial protrusion or radial recess to engage with a complementary shaped fastening element 53 as provided on an outside surface of the insert 52. This way, the proximal end 44 of the cartridge holder 31 can be fixed and attached to the body 6.

Optionally, the proximal end 44 of the cartridge holder 31 comprises a coding element 54 by way of which the cartridge assembly 150 can be mechanically encoded to distinguish from another cartridge assembly 150, e.g. equipped with a different type of cartridge 10 or with a cartridge containing a different medicament 26. The coding element 54 or coding feature may be complementary shaped or complementary positioned to a counter coding element 84 as provided on or inside the body 6. A non-matching pair of a coding element 54 and a counter coding element 84 may prevent a mutual fastening of the cartridge holder 31 to the body 6.

A mutual fastening and fixing of the cartridge holder 31 to the body 6 may be only provided and allowed when the coding element 54 of the cartridge holder 31 matches in size, shape and position with the corresponding counter coding element 84 as provided on or inside the body 6.

In one aspect the present disclosure relates to a cartridge assembly 150 comprising a housing component 30, e.g. implemented as a cartridge holder 31, and further comprising a cartridge 10 and a fixing member 100.

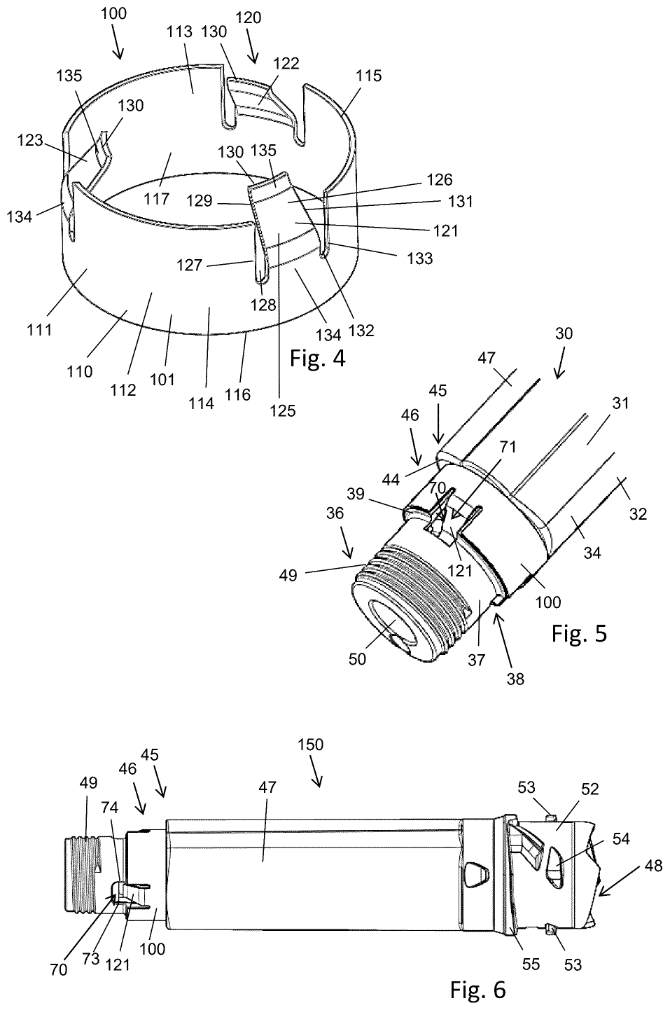

The fixing member 100 as illustrated in FIG. 4 comprises a fastening portion 110 and a fixing structure 120. The fastening portion 110 is configured for fastening to a complementary-shaped counter fastening portion 60 as provided on an outside surface 34 of the housing sidewall 32 of the housing component 30. As it is apparent from FIGS. 1-6 the counter fastening portion 60 is provided on a neck portion 46 of the cartridge holder 31. The counter fastening portion 60 comprises a sidewall portion 62 of tubular shape. An outside surface 63 of the sidewall portion 62 comprises a gliding surface 67, allowing for a rather smooth sliding or gliding of the complementary shaped inside surface 113 of the fastening portion 110 of the fixing member 100 in the course of assembling the fixing member 100 on the cartridge holder 31. Insofar, the inside surface 113 of the sidewall 111 of the body 101 of the fixing member 100 may also provide a gliding surface 117 complementary shaped to the gliding surface 67 of the counter fastening portion 60 of the cartridge holder 31.

In the presently illustrated example the fixing structure 120 comprises a first fixing element 121, a second fixing element 122 and a third fixing element 123. The fixing element 121, 122, 123 are equidistantly separated along the circumference of the fixing member 100. The body 101 of the fixing member 100 is of somewhat tubular shape. The fastening portion 110 comprises a tubular-shaped sidewall 111 forming a fastening ring 112. The sidewall 111 is of annular and/or tubular shape. The fastening ring 112 comprises a closed ring structure. With other examples (not illustrated) the fastening ring may comprise an open or interrupted annular structure, by way of which an elasticity of the fixing member could be increased.

With some examples the fixing member 100 is made of a sheet metal. It may be unitarily formed. It may comprise a single piece. The individual fixing elements 121, 122, 123 each comprise or constitute a snap element 125, which is resiliently deformable in radial direction. The snap element 125 and hence the fixing elements 121, 122, 123 each comprise a longitudinally extending tongue portion 126 protruding radially inwardly from the sidewall 111 of the body 101 of the fixing member 100. The tongue portions 126 each comprise a bottom portion 134 by way of which the tongue portions 126 are connected to the annular shaped fastening portion 110. When correctly assembled to the housing component 30, the tongue portions 126 extend or protrude in distal direction 2 from the fastening portion 110.

Hence, a free end section as provided by a head portion 135 forming the opposite end of the bottom portion 134 of the tongue portions 126, is located closer to the distal end 42 of the cartridge holder 31 than the bottom portion 134.

The bottom portion 134 may longitudinally adjoining the fastening portion 110. An outside surface of the bottom portion 134 may flush with the outside surface 114 of the fastening portion 110. With some examples it is even conceivable that the tongue portion 126 comprises a S-like shape. Here, the bottom portion 134 may protrude radially outwardly from the outside surface 114 of the fastening portion 110. A middle section and/or an oppositely located head portion 135 of the tongue portion 126 may then protrude radially inwardly. A S-shaped structure of the tongue portion 126 may provide an increased biasing effect and may be beneficial to provide a resilient clamping effect when attached to the outside surface 34 of the cartridge holder 31.

With some examples and as illustrated in FIG. 4 the tongue portions 126 are of somewhat rectangular or tapered shape. While the bottom portion 134 is integrally formed with the body 101 of the fixing member 100 longitudinally extending oppositely located side edges 129, 131 are separated from the sidewall 111 of the fixing member 100 by longitudinal slit 128, 132.

Here, the sidewall 111 comprises a longitudinally extending or axially extending side edge 127 adjacent to a slit 128. The opposite side of the slit 128 is confined by a longitudinally extending side edge 129 of the tongue portion 126. An oppositely located circumferential end of the tongue portion 126 is delimited by the side edge 131, which adjoins the slit 132. The slit 132 separates the side edge 133 of the sidewall 111 from the side edge 131 of the tongue portion 126. In this way and due to the inherent elasticity of the material of the body 101 of the fixing member 100 at least the free end and hence the head portion 135 of the tongue portion 126 is radially deformable and can be urged or moved radially outwardly against a restoring force.

In the initial configuration as illustrated in FIG. 4, the head portions 135 of the tongue portions 126 protrude radially inwardly. They protrude radially inwardly from an inside surface 113 of the sidewall 111.

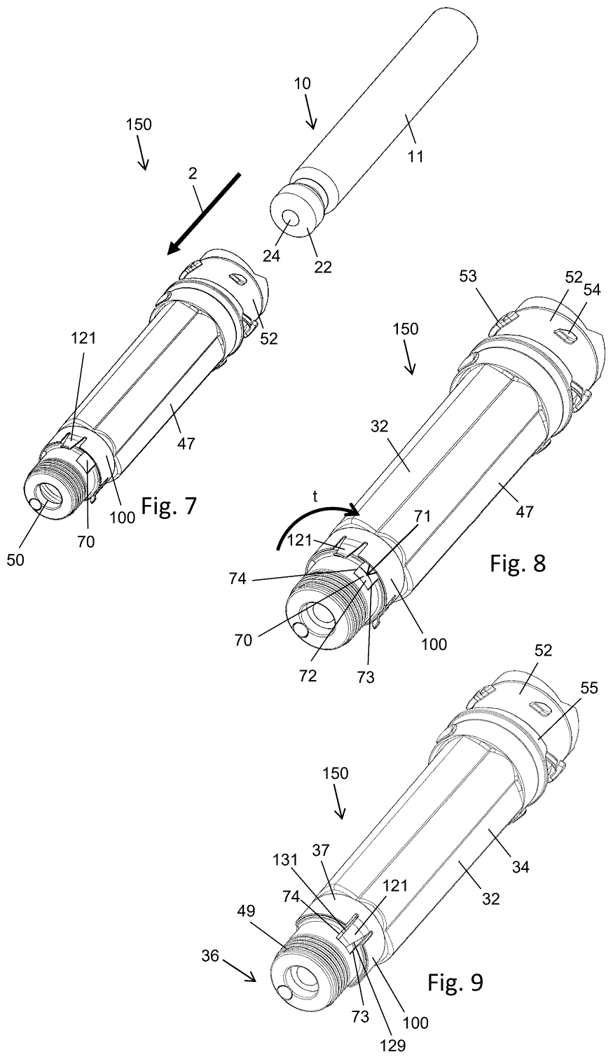

The fixing member 100 is provided as a separate piece that is suitable for attachment and/or fixing to the cartridge holder 31, e.g. after insertion of the cartridge 10 into the cartridge receiving space 33. As illustrated in greater detail in FIGS. 1-6 the housing sidewall 32 of the cartridge holder comprises a counter fixing structure 70. With the example of FIGS. 1-12 the counter fixing structure 70 comprises a through opening 72 extending through the housing sidewall 32. The through opening 72 forms a fixing recess 71 to receive the fixing element 121 of the fixing structure 120 of the fixing member 100 when the fixing member 100 reaches the final assembly configuration.

As illustrated in FIGS. 1-12 the first fixing element 121 snaps into the fixing recess 71 and extends through the through opening 72 into the interior of the container receiving space 33. As it is particularly illustrated in FIG. 11 and when reaching a final assembly configuration the first fixing element 121 engages the radially recessed portion 16 of the cartridge 10. In detail, a distally facing stop face 130 of the tongue portion 126 gets in longitudinal abutment with a proximally facing counter stop face 28, e.g. provided at or on the radially widened head portion 18 of the cartridge 10.

As shown in FIG. 11 the counter stop face 28 may be covered or provided by the septum retainer 22. In the final assembly configuration, the fastening portion 110 of the fixing element 100 is in longitudinal abutment with a distally facing stop face 44 as provided on the outside surface 34 of the sidewall housing 32 of the cartridge holder 31. The stop face 44 is provided at or on the shoulder portion 45 of the housing sidewall 32. It provides a proximal stop for the fastening portion 110, e.g. when the fixing member 100 is mounted onto the cartridge holder 31 in proximal direction 3. Here, a proximal end face 116 of the sidewall 111 of the fastening portion 110 gets in a longitudinal and hence proximal abutment with the distally facing stop face 44 as provided on the cartridge holder 31, thereby limiting a proximally directed assembly motion of the fixing member 100 relative to the cartridge holder 31.

Since the stop face 130 of the first fixing element 121 is in a longitudinal abutment with the proximally facing counter stop face 28 of the cartridge 10 when in the final assembly configuration as illustrated in FIG. 11, any attempts to remove the cartridge 10 towards the proximal direction 3 lead to a transfer of a respective force onto the fixing element 121, which in turn cannot move in proximal direction 3 due to the longitudinal abutment of the sidewall 111 at the stop face 44 of the cartridge holder 31.

As further illustrated in FIG. 11 the counter fastening portion 60 of the cartridge holder 10 is confined in distal direction by a radially stepped down portion 39, which adjoins a connector-freeportion 37 of the needle connector 36 at the distal end 42 of the cartridge holder 31. In the final assembly configuration, the distal end face 115 of the sidewall 111 of the fixing member 100 is aligned with the stepped portion 39. In this way, almost the entirety of the inside surface 113 of the sidewall 111 of the fixing member 100 may be in mechanical contact with the complementary shaped outside surface 63 of the counter fastening portion 60. In this way there can be provided a rather robust and well-defined mechanical support for the fixing member 100 on the outside surface 34 of the housing sidewall 32.

With the fixing element 121 radially inwardly protruding through the through opening 72 of the housing sidewall 32 and longitudinally engaging with the recessed neck portion 16 of the cartridge 10 there can be also provided a non-releasable fixing of the fixing member 100 to the cartridge holder 31. As shown in detail in FIG. 11 the distal end face 29 of the cartridge 10, e.g. the distal end face of the septum retainer 22, is in longitudinal abutment with a proximally facing portion of an end wall 57 of the distal end 42 of the cartridge holder 10.

When in the final assembly configuration the distal end of the cartridge 10 is in longitudinal abutment with a proximally facing side of the end wall 57. When in the final assembly configuration the cartridge 10 cannot be moved any further in distal direction 2 relative to the cartridge holder 31. Now and when the fixing structure 120, in particular the fixing element 121 is in longitudinal abutment with the counter stop face 28 as illustrated in FIG. 11, the fixing member 100 is hindered to move in distal direction 2 relative to the cartridge 10 and hence relative to the cartridge holder 31 because the cartridge 10 is in distal abutment with the end wall 57.

In this way, the mutual engagement of the fastening element 121 with the radially recessed portion 16 of the cartridge inside the container receiving space 33 serves to fix the cartridge 10 inside the cartridge holder 31 and also prevents a removal of the fixing member 100 from the cartridge holder 31 in distal direction 2. For removing the fixing member there may be required a special tool by way of which the fixing element 121 is urged or moved radially outwardly thereby disengaging from the neck portion 16 or head portion 18.

Thereafter and when urging the head portion 135 of the tongue portion 126 radially outside of the counter fixing structure 70 the cartridge 10 may be removed from the cartridge holder 31 in proximal direction 3 and/or the fixing member 100 may be removed from the cartridge holder in a distal direction 2.

With some examples and when the through opening 72 comprises a geometry that closely matches the geometry of the fixing element 121, 122, 123 it may be somewhat impossible to enter the through opening 72 with a tool for urging any of the fixing elements 120 121, 122, 123 radially outwardly, e.g. for disengaging from the cartridge 10. This may particularly apply when the longitudinal side edges 73, 74 of the through opening 72 comprise a circumferential distance that is equal than or only slightly larger than a circumferential distance between the oppositely located side edges 129, 131 of the tongue portion 126.

There may be provided further examples of the cartridge assembly 150, wherein the circumferential distance between the longitudinally extending side edges 73, 74 of the counter fixing structure 70 is generally larger than the respective circumferential extent of the tongue portion 126. Then, establishing of a final assembly configuration and hence establishing of a snap in of the fixing element 121, 122, 123 into respective fixing recesses 71 or through openings 72 can be facilitated. Likewise, also a disassembly could be enabled, e.g. by making use of a tool sized to enter into the trough opening 72 and configured to bend or to move the fixing elements 121, 122, 123 radially outwardly.

With the sidewall 111 of the fastening portion 110 or fixing member 100 terminating in distal direction 2 with the stepped portion 39 of the housing sidewall 32 the fixing member 100 can be entirely located outside the needle connector 36 or outside the connector-free portion of the housing sidewall 32, thus allowing and supporting a rather easy and smooth attachment of a needle assembly to the needle connector 36.

With the example of the cartridge assembly 150 as illustrated in FIGS. 1-6 it is intended that the cartridge 10 is arranged inside the cartridge or container receiving space 33 before the fixing member 100 is arranged in the final assembly configuration as illustrated in FIG. 5. As indicated in FIG. 3, the fixing member 100 may be attached or arranged on the outside surface 34 of the cartridge holder 31 after insertion of the cartridge 10 into the cartridge receiving space 33. Attachment of the fixing member 100 to the counter fastening portion 60 of the cartridge holder 31 may include a longitudinal sliding motion of the fixing member 100 relative to the cartridge holder 31 in proximal direction 3.

A longitudinal sliding motion may require a proper circumferential alignment of the fixing element 121 with the counter fixing structure 70 as provided on the outside surface 63 of the counter fastening portion 60. When reaching the final assembly configuration, the radially inwardly extending tongue portions 126 of the various fixing element 121, 122, 123 snap into the fixing recesses 71 and may hence extend through the respective through opening 72 as provided on or in the neck portion 46 of the cartridge holder 31.

In a preassembly configuration (not illustrated) the fixing member 100 may be arranged or attached to the neck portion 46 of the cartridge holder 31 before or during insertion of the cartridge holder 10. Here, the fixing member 100 may be arranged in a preassembly configuration, which is distally offset from the final assembly configuration as shown in FIG. 5. In such a preassembly configuration the inside of the radially inwardly biased fixing element 121, 122, 123 may be in engagement with the needle connector 36, in particular with the threaded section 49 or with the connector-free portion 37 of the needle connector 36. In this way and by the radially inwardly biased configuration of the fixing element 121, 122, 123 the fixing member 100 can be kept in a stable preassembly configuration. This kind of pre-fixing to the housing component 30 is beneficial to avoid a separate handling of the fixing member, e.g. in the course of assembly of the cartridge assembly 150 or of a respective drug delivery device 1.

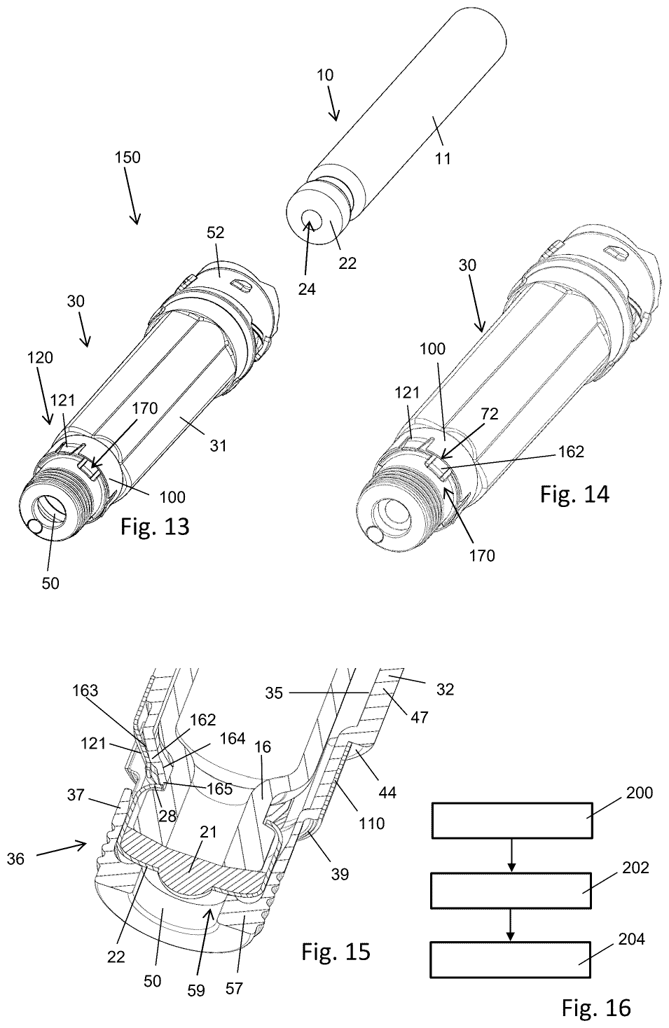

In the example of FIGS. 13-15 the fixing member 100 is shown in another preassembly configuration. Here, the preassembly position or preassembly configuration of the fixing member 100 is circumferentially offset from the final assembly configuration. The fixing structure 120 and hence the fixing element 121 is at a certain angular offset from the position of the counter fixing structure 170. For transferring the fixing member 100 from the preassembly configuration as shown in FIGS. 13 and 14 into a final assembly configuration it is required to rotate the fixing element 100 clockwise relative to the cartridge holder 31 and hence along a tangential direction t as illustrated by the arrow in FIG. 8. When reaching a final assembly configuration the fixing element 121 snaps into the fixing recess 71 one and provides a likewise longitudinal fixing of the cartridge 10 inside the cartridge receiving space 33 as described above in connection with FIGS. 1-6.

Once the fixing element 121 engages the counter fixing structure 70 the side edges 129, 131 of the tongue portion 126 collide with the side edges 73, 74 of the through opening 72 when a user should attempt to rotate the fixing member 100 relative to the cartridge holder 31 after establishing of the final assembly configuration. Insofar, the engagement of the fixing structure 120 with the counter fixing structure 70 provides a rotational interlock for non-rotationally fixing the fixing member 100 to the cartridge holder 31.

With the further example as illustrated in FIGS. 13-15 the counter fixing structure 170 is slightly modified compared to the counter fixing structure 70 as described before. Here, the counter fixing structure 170 comprises a flap portion 162, which is integrally formed with the sidewall 32 of the cartridge holder 31. The flap portion 162 is deformable or bendable in radial direction. The flap portion 162 may cover at least a portion of a through opening 72 provided in the neck portion 46 of the sidewall 32 of the cartridge holder 31. The flap portion 162 may be integrally attached to the sidewall 32. Typically, a proximal end of the flap portion 162 may be integrally formed with the sidewall 32. An opposite end, hence a distal end of the flap portion 162 is typically movable in radial direction so as to engage with the counter stop face 28 of the cartridge 10 as illustrated in FIG. 15.

Here, the flap portion 162 comprises a distally facing stop face 165 to longitudinally engage or abut with the proximally facing counter stop face 28 of the cartridge 10. The flap portion 162 comprises an outside surface 163, which may engage with the inside surface of the fastening element 121. In this way and when correctly aligned with the counter fixing structure 177 the fixing structure 120, in particular the fixing element 121 serves to apply a radially inwardly directed force effect onto the movable or radially deformable flap portion 162, thereby urging the flap portion 162 radially inwardly.

Then, the stop face 165 of the flap portion 162 engages the recessed neck portion 16 of the cartridge 10. Moreover, an inside surface 164 of the flap portion 162 may get in mechanical contact with an outside surface of the barrel 11 of the cartridge 10. Typically, the flap portion 162 is made of a plastic material. It may be implemented as an integral component of the cartridge holder 31, which may be implemented as an injection molded plastic component. The fixing member 100 may be implemented as a plastic component or as a metal component. When the fixing member is implemented as a metal component the flap portion 162 prevents a direct contact between the metal part of the fixing member 100 with the cartridge 10.

With the implementation of the counter fixing structure 170 as shown in FIGS. 13-15 it is also conceivable to arrange the fixing member 100 in the final assembly configuration before the cartridge 10 is inserted into the cartridge receiving space 33. Here, the flap portion 162 may protrude radially inwardly from an inside surface 35 of the sidewall 32 and hence into the cartridge receiving space 33. Since the flap portion 162 is made of a rather soft or pliable material there may not arise a significant damage to the cartridge 10 or to the septum retainer 22 when the cartridge is urged in distal direction 2 into the final assembly configuration inside the cartridge receiving space 33, thereby at least temporally urging the radially biased flap portion 162 radially outwardly in the course of cartridge assembly.

Then and when the cartridge 10 reaches the final assembly configuration, i.e. when the distal end face 29 of the cartridge 10 abuts an inside facing portion of the end wall 57 the radially widened head portion 18 will be located distally offset from the flap portion 162, thereby allowing that the flap portion 162 is urged radially inwardly under the effect of the relaxing fixing element 121.

The flowchart of FIG. 16 is illustrative of a method of assembling a cartridge assembly 150 as described herein. In a first step 200 a cartridge 10 is inserted into the cartridge receiving space 33 of a cartridge holder 31. Typically, the cartridge 10 is inserted along the longitudinal distal direction 2. It is placed into the cartridge receiving space 33 via the insert opening 48 provided at the proximal end 43 of the cartridge holder 31.

Thereafter and in step 202 the fixing member 100 is mounted on the cartridge holder 31. During step 202 the fixing member may be placed or moved towards and into a final assembly configuration, wherein the fastening portion 110 of the fixing member 100 engages the counter fastening portion 60 as provided on the outside surface 34 of the housing sidewall 32 of the cartridge holder 31. In step 200 the fixing member 100 may be moved from a preassembly configuration, in which the fastening portion 110 is already engaged with the counter fastening portion 60, into the final assembly configuration, in which the fixing structure 110 engages the counter fixing structure 170.

The final step of assembly is reached with step 204. Here, and upon reaching the final assembly configuration the fixing element 121 snaps into the counter fastening structure 70 and may thus extend through a through opening 72 into the cartridge receiving space 33, thereby engaging e.g. with the a recessed neck portion 16 of the cartridge 10.

With another example the step of inserting the cartridge 10 and the step of mounting the fixing member 100 on the cartridge holder 31 may be interchanged. Then, the fixing member 100 may be arranged in a final assembly position on the cartridge holder 31 before the cartridge 10 is inserted into the cartridge receiving space 33. In a subsequent step the cartridge 10 is urged into the final assembly position inside the cartridge receiving space 33, thereby at least temporally urging the radially inwardly biased fastening elements 121, 122, 123 radially outwardly. When reaching the final assembly configuration, the fastening elements 121, 122, 123 relax radially inwardly, thereby engaging the radially recessed neck portion 16 of the cartridge.

### REFERENCE NUMBERS

1 drug delivery device
2 distal direction
3 proximal direction
4 protective cap
5 medicament container
6 body
7 drive mechanism
8 dose member
9 trigger
10 cartridge
11 barrel
12 sidewall
14 main portion
15 shoulder portion
16 neck portion
18 head portion
20 seal
21 septum
22 septum retainer
23 beaded cap
24 aperture
25 piston
26 medicament
27 dispensing end
28 counter stop face
29 distal end face
30 housing component
31 cartridge holder
32 sidewall
33 cartridge receiving space
34 outside surface
35 inside surface
36 needle connector
37 connector-free portion
38 cylindrical portion
39 stepped portion
42 distal end
43 proximal end
44 stop face
45 shoulder portion
46 neck portion
47 main portion
48 insert opening
49 threaded section
50 aperture
52 insert
53 fastening element
54 coding element
55 flange portion
57 end wall
60 counter fastening portion
62 sidewall portion
63 outside surface
67 gliding surface
70 counter fixing structure
71 fixing recess
72 through opening
73 side edge
74 side edge
80 piston rod
81 pressure piece
82 receptacle
83 counter fastening structure
84 counter coding element
100 fixing member
101 body
110 fastening portion
111 sidewall
112 fastening ring
113 inside surface
114 outside surface
115 distal end face
116 proximal end face
117 gliding surface
120 fixing structure
121 fixing element
122 fixing element
123 fixing element
125 snap element
126 tongue portion
127 side edge
128 slit
129 side edge
130 stop face
131 side edge
132 slit
133 side edge
134 bottom portion
135 head portion
162 flap portion
163 outside surface
164 inside surface
165 stop face
170 counter fixing structure

1. A fixing member for fixing a medicament container inside a housing component of a drug delivery device, wherein the housing component comprises a housing sidewall confining a container receiving space and extending in a longitudinal direction (z), the fixing member comprising:
a fastening portion comprising an inside surface complementary shaped to an outside surface of a counter fastening portion of the housing sidewall, wherein the inside surface is sized and shaped to support at least one of a longitudinally directed and a circumferentially directed fastening motion of the fastening portion relative to the counter fastening portion for reaching a final assembly configuration, and
a fixing structure connected to the fastening portion and comprising a first fixing element, wherein the first fixing element is configured to extend into the container receiving space to mechanically engage with the medicament container located inside the container receiving space when the fastening portion reaches the final assembly configuration on the counter fastening portion.

2. The fixing member according to claim 1, wherein the fastening portion comprises a sidewall of annular shape.

3. The fixing member according to claim 1, wherein the fastening portion comprises an annular shaped fastening ring.

4. The fixing member according to claim 1, wherein the fastening portion comprises a closed ring.

5. The fixing member according to claim 1, wherein the inside surface comprises a gliding surface configured to glide on a gliding surface of the outer surface of the counter fastening portion along at least one of the longitudinal direction (z) and the circumferential direction (t).

6. The fixing member according to claim 1, wherein the first fixing element is at least one of resiliently movable or resiliently deformable relative to the fastening portion with regard to a radial direction (r).

7. The fixing member according to claim 6, wherein the first fixing element is biased radially inwardly and wherein the fixing element is resiliently movable or resiliently deformable radially outwardly against a restoring force.

8. The fixing member according to claim 1, wherein the first fixing element protrudes radially inwardly from the inside surface of the fastening portion.

9. The fixing member according to claim 1, wherein the first fixing element comprises a snap element with a tongue portion extending in the longitudinal direction (z).

10. The fixing member according to claim 9, wherein the tongue portion comprises a bottom portion and a head portion, wherein the bottom portion and the head portion are separated along the longitudinal direction (z), wherein the bottom portion is connected to the fastening portion and wherein the head portion forms a free end section of the tongue portion.

11. The fixing member according to claim 1, wherein the first fixing element comprises a distally facing stop face to engage with a proximally facing counter stop face of the medicament container.

12. The fixing member according to claim 1, wherein the first fixing element comprises an inside facing surface to engage with an outside surface of a flap portion of a counter fixing structure of the housing sidewall, wherein the flap portion is at least one of movable or deformable in radial direction and wherein the first fixing element is configured to urge the flap portion radially inwardly.

13. The fixing member according to claim 1, wherein the first fixing element comprises a side edge extending in longitudinal direction (z) and protruding at least in sections from the inside surface of the fastening portion to engage with a recessed counter fixing structure on the outside surface of the housing sidewall.

14. The fixing member according to claim 1, wherein the fixing structure further comprises a second fixing element and/or a third fixing element located circumferentially offset from the first fixing element and configured to extend into the container receiving space to mechanically engage with the medicament container inside the container receiving space.

15. A cartridge assembly for a drug delivery device, comprising:
a cartridge comprising a tubular shaped barrel containing a medicament, wherein the cartridge comprises a radially recessed neck portion,
a housing component of the drug delivery device, the housing component comprising:
a housing sidewall confining a cartridge receiving space extending in a longitudinal direction and sized to receive the cartridge,
a counter fastening portion provided on an outside surface of the housing sidewall, the cartridge assembly further comprising:
a fixing member according to claim 1, wherein the fastening portion of the fixing member is mechanically engageable with the counter fastening portion and wherein the first fixing element of the fixing structure of the fixing member extends into the container receiving space and mechanically engages with the neck portion of the cartridge when the fastening portion of the fixing member is mechanically engaged with the counter fastening portion.

16. The cartridge assembly according to claim 15, wherein the housing sidewall comprises a counter fixing structure with a first through opening extending through the housing sidewall and sized to receive the first fixing element radially therein or therethrough.

17. The cartridge assembly according to claim 15, wherein the housing sidewall comprises a counter fixing structure with a first flap portion movable or deformable in radial direction (r) relative to the housing sidewall, wherein the first flap portion comprises an outside surface to engage with the first fixing element of the fixing structure of the fixing member and further comprises an inside surface to engage with the neck portion of the cartridge.

18. The cartridge assembly according to claim 15, wherein the counter fastening portion comprises a tubular-shaped sidewall portion with an outer gliding surface enabling or supporting at least one of a longitudinal and a circumferential sliding motion of the fastening portion of the fixing member relative to the housing sidewall.

19. A drug delivery device for dispensing of a dose of a medicament, the drug delivery device comprising:
a body,
a drive mechanism comprising a piston rod to exert a dispensing force onto a piston located inside a cartridge containing the medicament, and
a cartridge assembly according to claim 15 any one of the preceding claims 15 to 18 and being configured to releasably or non-releasably fix to the body.

20. (canceled)

21. A method of assembling a cartridge assembly of a drug delivery device, the method comprising the steps of:
inserting a cartridge into a cartridge receiving space of a housing component of a cartridge assembly;
arranging a fixing member according to claim 1 in a final assembly position on the counter fastening portion of the housing sidewall of the housing component, and
mechanically engaging the first fixing element of the fixing structure of the fixing member with a neck portion of the cartridge.
